(19) United States
(12) Patent Application Publication
(10) Pub. No.: US 2026/0255070 A1
(43) Pub. Date: Aug. 27, 2026

(54) MEDICAL DEVICES AND RELATED SYSTEMS AND METHODS FOR REDUCING SIGNAL DISTORTION IN IMAGE SIGNALS

(71) Applicant: Boston Scientific Scimed, Inc., Maple Grove, MN (US)

(72) Inventors: George Wilfred DUVAL, Sudbury, MA (US); Louis J. BARBATO, Franklin, MA (US)

(73) Assignee: Boston Scientific Scimed, Inc., Maple Grove, MN (US)

(21) Appl. No.: 19/649,625

(22) Filed: Apr. 16, 2026

Related U.S. Application Data
(63) Continuation of application No. 18/785,148, filed on Jul. 26, 2024, now abandoned.
(60) Provisional application No. 63/515,857, filed on Jul. 27, 2023.

(51) Int. Cl. H04N 23/80; A61B 1/00; A61B 1/045; H04N 23/50

(52) U.S. Cl. CPC H04N 23/80; A61B 1/000095; A61B 1/00016; A61B 1/00045; A61B 1/045; H04N 23/555

(57) ABSTRACT

According to one aspect, a medical device system for visualizing internal patient anatomy may comprise: a shaft including a distal tip portion, the tip portion including an imaging device, and a signal modulator; and a control unit operatively coupled to the shaft and including a de-modulator. The imaging device may be configured to output a first signal to the signal modulator. The signal modulator may be configured to modulate the received first signal and output a modulated second signal to the control unit; and the de-modulator of the control unit may be configured to receive the modulated second signal, de-modulate the second signal, and output a de-modulated third signal. The control unit may be configured to output the de-modulated third signal to an electronic display.

### CROSS-REFERENCE TO RELATED APPLICATIONS

This application is a continuation of U.S. Patent Application No. 18/785,148, filed on July 26, 2024, which claims the benefit of priority of U.S. Provisional Patent Application No. 63/515,857, filed on July 27, 2023, each of which is incorporated by reference herein in its entirety.

### TECHNICAL FIELD

Various aspects of this disclosure relate generally to medical devices, particularly scopes such as endoscopes or bronchoscopes, including imaging elements. More specifically, embodiments of this disclosure relate to reducing noise and other signal distortion in image signals from an imager in an endoscope or other medical device, among other aspects.

### BACKGROUND

Endoscopes have attained great acceptance within the medical community since they provide a means for performing procedures with minimal patient trauma while enabling the physician to view the internal anatomy of the patient. Numerous endoscopes have been developed and categorized according to specific applications, such as cystoscopy, colonoscopy, laparoscopy, upper GI endoscopy, and others. Endoscopes may be inserted into the body's natural orifices or through an incision in the skin.

An endoscope is usually an elongated tubular shaft, rigid or flexible, having a video camera or a fiber optic lens assembly at its distal end. The shaft is connected to a handle. Viewing is usually possible via an external screen. Various surgical tools may be inserted through a working channel in the endoscope for performing different surgical procedures. Endoscopes, such as colonoscopes, that are currently being used typically have a front camera for viewing the internal organ, such as the colon, an illuminator, a fluid injector for cleaning the camera lens (and sometimes also the illuminator), and a working channel for insertion of surgical tools, for example, for removing polyps found in the colon. Often, endoscopes also have fluid injectors ("jets") for cleaning a body cavity, such as the colon, into which they are inserted. The illuminators commonly used are fiber optics which transmit light, generated remotely, to the endoscope tip section and light-emitting diodes (LEDs) at the endoscope tip section.

Current endoscopes typically implement an imaging system through a camera sensor at a tip section of the endoscope, which is commonly a charge coupled device (CCD) sensor or a complementary metal-oxide semiconductor (CMOS) sensor. Some cameras combine two or more sensor arrays to improve sharpness and performance. However, image quality is not only linked to camera sensors. The whole imaging chain has to be optimally concerted. This includes the lenses, endoscope optics, image signal transfer means, processing systems, the monitor and other components. Additionally, the image documentation mode (e.g., video or static images) plays an important role and can influence the diagnostic value.

The acquired camera sensor data is transferred to a video processing unit for conversion into an image. The video processor optimizes the image or real-time video depending on the selected preset settings like white balance, color display mode, reflection reduction or image rotation, and the video processor transfers the image or real-time video to a screen. Additionally, scenes and images can be stored for later diagnostics or documentation. Some video processors provide, in combination with distinct light sources, enhancement technologies like narrow band imaging (NBI), autofluorescence (AF) or flexible spectral imaging color enhancement (FICE).

The raw analog signal data sent from the camera sensor at the tip of the endoscope may have to travel a number of feet before the signal is processed by a control unit, for example may have to travel 5 or more feet over the length of an endoscope shaft and through an endoscope’s umbilicus cord. In smaller imagers for smaller diameter endoscopes, such as bronchoscopes or cholangioscopes, the camera sensor data acquired may be a raw analog signal without any pre-processing done at the tip portion of the scope. This means that the raw analog signal is more susceptible to picking up noise while running the length of the endoscope and potentially through the umbilicus cord too. Also, other environmental considerations may introduce additional noise to the signal.

There is a need in the art for image processing devices, systems, and methods that may be implemented within the size and hardware limitations of medical devices, such as endoscopes and particularly smaller-diameter endoscopes, and which also provide reduced noise in a camera signal.

### SUMMARY

Aspects of the disclosure relate to, among other things, systems, devices, and methods for reducing noise in an image signal of a medical device, among other aspects. The systems, devices, and methods of this disclosure may help to reduce noise in a raw or processed image signal received from a distal tip portion of an endoscope or other medical device. The systems, devices, and methods of this disclosure may reduce the need for pre-processing of an image signal at a distal tip potion of a medical device, may facilitate reducing the size of a tip portion of an endoscope, may increase clarity in medical images from endoscopes, and may help address other issues. Each of the aspects disclosed herein may include one or more of the features described in connection with any of the other disclosed aspects.

According to one aspect, a medical device system for visualizing internal patient anatomy may comprise: a shaft including a distal tip portion, the tip portion including an imaging device, and a signal modulator; and a control unit operatively coupled to the shaft and including a de-modulator. The imaging device may be configured to output a first signal to the signal modulator. The signal modulator may be configured to modulate the received first signal and output a modulated second signal to the control unit; and the de-modulator of the control unit may be configured to receive the modulated second signal, de-modulate the second signal, and output a de-modulated third signal. The control unit may be configured to output the de-modulated third signal to an electronic display.

In other aspects, the medical device system may include one or more of the following features. The second signal may be transferred to the de-modulator via a single wire. The distal tip portion may further comprise a low pass filter. The distal tip portion may further comprise a pulse generator. The distal tip portion may further comprise a pulse reshaping circuit. The control unit may comprise a holding circuit configured to receive the second signal. The control unit may further comprise a low-pass filter configured to receive the second signal from the holding circuit. The medical device may be an endoscope. The second signal may be transferred to the de-modulator via an antenna. The modulator may be configured to apply a carrier technique to the first signal to create the second signal, and the carrier technique may include at least one of: amplitude modulation (AM), pulse-amplitude modulation (PAM), pulse-width modulation (PWM), frequency modulation (FM), or phase modulation (PM). The modulator may be configured to apply a frequency modulation carrier technique and output a second signal at 24 Megahertz bandwidth. The modulator may be configured to apply a pulse amplitude modulation carrier technique. The modulator may be configured to apply a phase modulation carrier technique. The first signal may have a frequency between 10 Megahertz and 99 Megahertz; and the modulator may be configured to apply a carrier pulse train with a frequency between (i) 2.5 times higher than the frequency of the first signal and (ii) 5 times higher than the frequency of the first signal. The second signal may be the integral of the first signal.

In other aspects, a medical device system for visualizing internal patient anatomy may comprise: a shaft including a distal tip portion, the tip portion including an imaging device, and a signal modulator; and a handle operatively coupled to the shaft and including a de-modulator. The imaging device may be configured to output a first signal to the signal modulator. The signal modulator may be configured to modulate the received first signal and output a modulated second signal to the control unit. The de-modulator of the handle may be configured to receive the modulated signal, de-modulate the second signal, and output a de-modulated third signal; and the handle may be configured to output the third signal to an electronic display or a control unit.

In other aspects, the medical device system may include one or more of the following features. The distal tip portion may comprises a low pass filter. The distal tip portion may comprise a pulse generator. The distal tip portion may comprise a pulse reshaping circuit.

In other aspects, a method of operating a medical device that includes a handle and a shaft extending longitudinally from the handle, the method comprising: receiving, at a modulator in a distal tip portion of the shaft, a first signal from an imaging device at the distal tip portion; sending a modulated second signal to a demodulator in the handle; and sending a demodulated third signal from the demodulator to a control unit.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIGS. 1A and 1B are perspective views of an exemplary endoscope, according to aspects of this disclosure.

FIG. 2 illustrates an exemplary signal pathway of a raw analog imager signal to a signal wire carrier, according to aspects of this disclosure.

FIG. 3 illustrates an exemplary signal pathway of a modulated image signal to a de-modulated signal, according to aspects of this disclosure.

FIGS. 4A-4C illustrate exemplary unmodified and modified signal waveforms, according to aspects of this disclosure.

FIG. 4D illustrates a waveform generated by a pulse width modulation technique, according to aspects of this disclosure.

FIG. 5 illustrates an exemplary signal pathway of a raw analog imager signal to a transmitter (e.g. single wire carrier), according to aspects of this disclosure.

FIG. 6 illustrates an exemplary imaging signal modulation technique for a medical device, according to aspects of this disclosure.

FIG. 7 is an example of ultra-high broadband phase modulation carrier technique with a high modulation index, according to aspects of this disclosure.

FIG. 8 illustrates an exemplary phase modulation scheme, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Throughout the figures included in this application, arrows labeled “P” and “D” are used to show the proximal and distal directions in the figure. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Embodiments of this disclosure may improve image quality of an image signal of a medical device, such as an endoscope, during a medical procedure and, as non-limiting exemplary benefits, help improve the visual display of a medical device’s camera or other imaging system, among other aspects. Embodiments of this disclosure may also specifically help to reduce noise and other signal distortion of an image signal of a medical device.

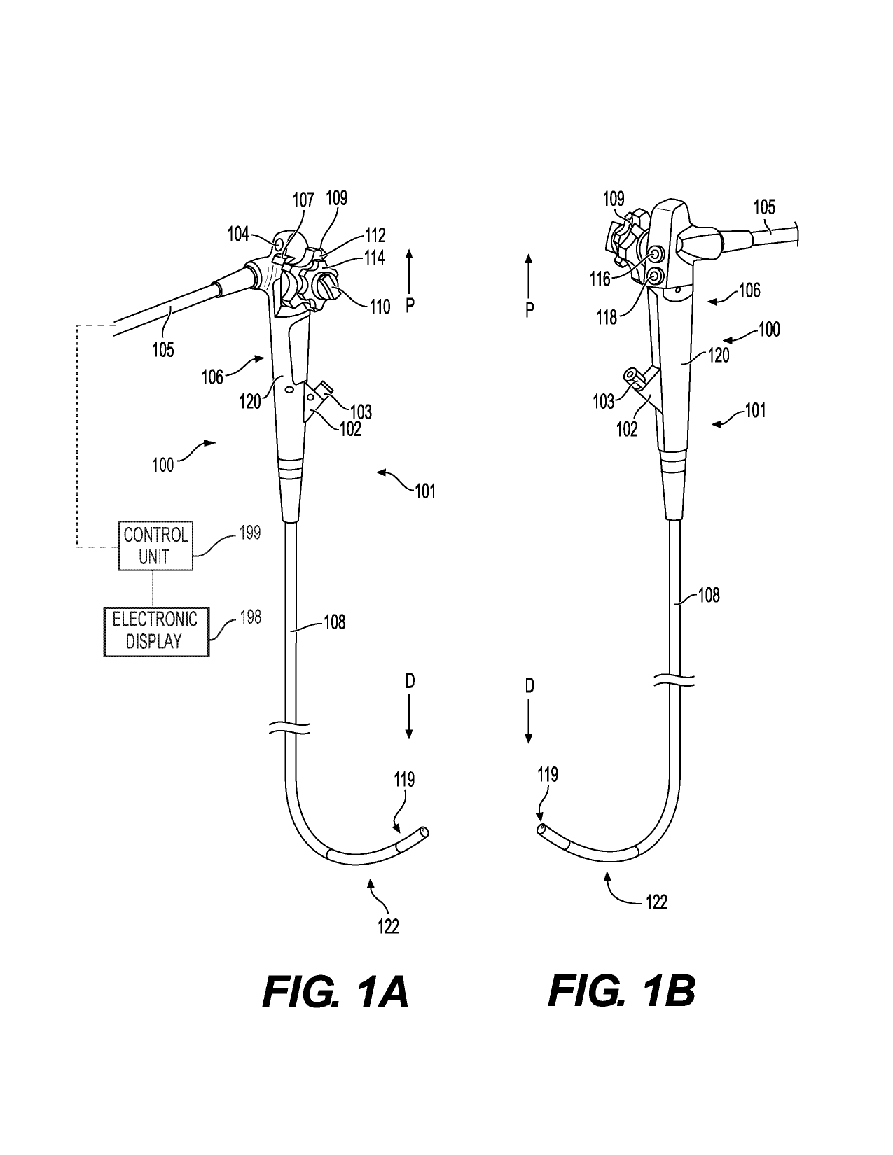

FIGS. 1A and 1B show perspective views of an exemplary endoscope system 100. Endoscope system 100 may include an endoscope 101. Endoscope 101 may include a handle assembly 106 and a flexible tubular shaft 108. The handle assembly 106 may include a biopsy port 102, a biopsy cap 103, an image capture button 104, an elevator actuator 107, a first locking lever 109, a second locking lever 110, a first control knob 112, a second control knob 114, a suction button 116, an air/water button 118, a handle body 120, and an umbilicus 105. All of the actuators, elevators, knobs, buttons, levers, ports, or caps of endoscope system 100, such as those enumerated above, may serve any purpose and are not limited by any particular use that may be implied by the respective naming of each component used herein. The umbilicus 105 may extend from handle body 120 to one or more auxiliary devices, such as a control unit 199, water/fluid supply, and/or vacuum source. Umbilicus 105 therefore may transmit signals between endoscope 101 and the control unit 199, in order to control lighting and imaging components of endoscope 101 and/or receive image data from endoscope 101. Umbilicus 105 also can provide fluid for irrigation from the water/fluid supply and/or suction to a distal tip 119 of shaft 108. Buttons 116 and 118 control valves for suction and fluid supply (e.g., air and water), respectively. Shaft 108 may terminate at distal tip 119. Shaft 108 may include an articulation section 122 for deflecting distal tip 119 in up, down, left, and/or right directions. Knobs 112 and 114 may be used for controlling such deflection, and locking levers 109 and 110 may lock knobs 112 and 114, respectively, in desired positions. Handle body 120 may be tapered and may narrow as the handle extends distally such that the profile of handle body 120 is smaller at its distal end than at its proximal end.

Although the term endoscope may be used herein, it will be appreciated that other devices, including, but not limited to, cholangioscopes, duodenoscopes, colonoscopes, ureteroscopes, bronchoscopes, laparoscopes, sheaths, catheters, or any other suitable delivery device or medical device may be used in connection with the devices of this disclosure, and the devices, systems, and methods discussed below may be incorporated into any of these or other medical devices.

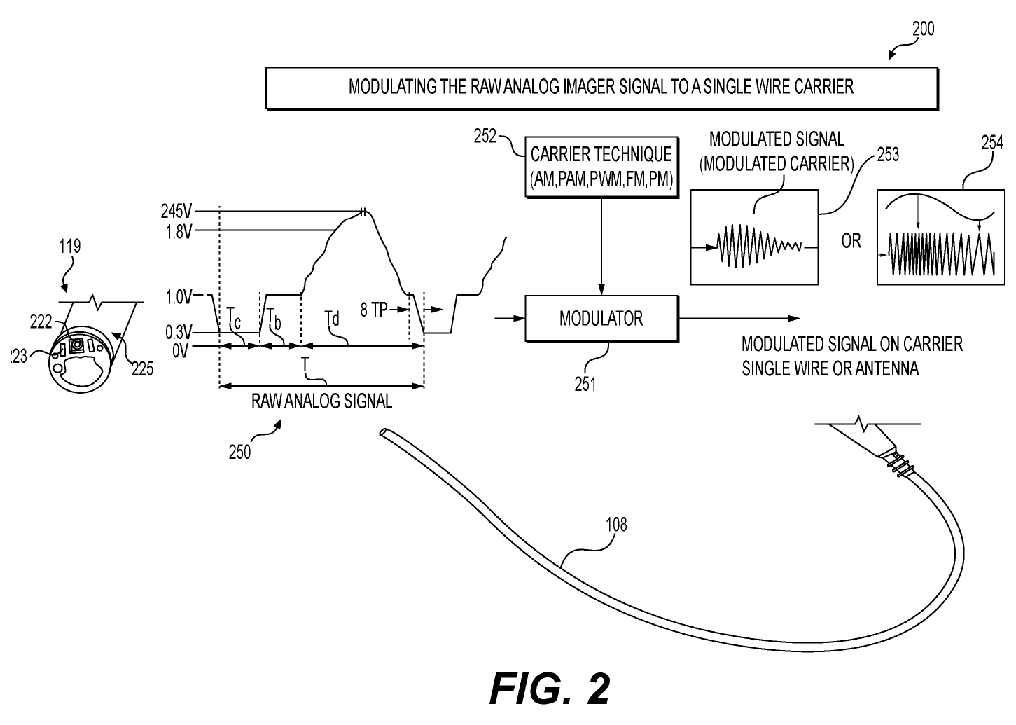

Distal tip 119 may include an imaging device 222 (e.g., an image sensor, camera, optical fiber, lens assembly with image sensor, etc.) and at least one lighting source 223 (e.g., an LED or an optical fiber), shown in FIG. 2. In some examples, distal tip 119 may include a front-facing imaging device 222 (as shown in FIG. 2), may include a front-facing imaging device 222 and a side-facing imaging device 222, may include only a side-facing imaging device 222, may include a front-facing imaging devices 222 and two side-facing imaging devices 222, or any other combination of imaging devices at distal tip 119. A side-facing imaging device 222 and the lighting source 223 may face radially outward, perpendicularly, approximately perpendicularly, or otherwise transverse to a longitudinal axis of shaft 108 and distal tip 119. A front-facing or forward-facing imaging device 222 or lighting source 223 may face approximately along or parallel to a longitudinal axis of distal tip 119 and shaft 108. In some examples, imaging device 222 may be a charge coupled device (CCD) image sensor, a complementary metal-oxide semiconductor (CMOS) image sensor, and/or any other element for converting light into electrons to be sent as an electrical signal.

Control unit 199 may be capable of interfacing with endoscope 101 to provide power and/or instructions for imaging device(s) 222 and/or light source 223. Control unit 199 may also control one or more other aspects of endoscope 101, such as, for example, the application of suction, the deployment or delivery of fluid, and/or the movement of distal tip 119. Control unit 175 may be powered by an external source such as an electrical outlet. In addition, the control unit 175 may include or otherwise be coupled to one or more buttons, knobs, touchscreens, or other user interfaces to control the imaging device 222, light source 223, and other features of endoscope 101. The control unit 199 may be housed in the handle 106 itself or in a separate apparatus.

Control unit 199 may be configured to enable the user to set or control one or more illumination and imaging parameters. For example, control unit 199 may enable the user to set or control an illumination level for each light source 223, gain level for each imaging device 222, exposure time for each imaging device 222, frame rate of each imaging devices 222, maximum or target values for any of the illumination and imaging parameters, and/or any other parameter associated with imaging device 222 and/or light source 223. In some examples, control unit 199 may be configured to execute one or more algorithms using one or more illumination and imaging parameters, for example to automatically adjust an illumination level of one or more of light sources 223 and/or automatically adjust one or more parameters of imaging device(s) 222. For example, control unit 199 may set or select an illumination level for one or more light sources 223 based on data received from one or more imaging devices 222. In some examples, as will be discussed in further detail below, control unit 199 may demodulate one or more image signals received from imaging device 222.

Control unit 199 may include electronic circuitry configured to receive, process, and/or transmit data and signals between endoscope 101 and one or more other devices. For example, control unit 199 may be in electronic communication with a display configured to display images based on image data and/or signals processed by control unit 199, which may have been generated by imaging device(s) 222 of endoscope 101. Control unit 199 may be in electronic communication with the display in any suitable manner, either via wires or wirelessly. The display may be manufactured in any suitable manner and may include touch screen inputs and/or be connected to various input and output devices such as, for example, mouse, electronic stylus, printers, servers, and/or other electronic portable devices. Control unit 199 may include software and/or hardware that facilitates operations such as those discussed above. For example, control unit 199 may include one or more algorithms, models, or the like for executing any of the methods and/or systems discussed in this disclosure. Control unit 199 may be configured to automatically adjust the illumination value applied to one or more light source(s) 223, and automatically adjust the gain and the frame rate applied to the one or more imaging device(s) 222.

In operating endoscope system 100, a user may use his/her left hand to hold handle assembly 106 (shown in FIG. 2A) while the right hand is used to hold accessory devices and/or operate one or more of the actuators of handle assembly 106, such as first and second control knobs 112, 114 and first and second locking levers 109, 110. The user may grasp the handle assembly 106 by wrapping the user’s hand around handle body 120. When grasping handle body 120, the user may use the left thumb to operate first and second control knobs 112, 114 and the elevator actuator 107 (through rotation about their respective axes), and may use a left-hand finger to operate the image capture button 104, the suction button 116, and/or the air/water button 118 (each by pressing). The user may rotate the handle assembly 106 (e.g., by moving his/her wrist) in order to rotate shaft 108 about a longitudinal axis of shaft 108 and position distal tip 119 at a target area within a patient’s body. The user may actuate button 104 to initiate video display from imaging device 222, take a photograph (such as a digital photograph) with imaging device 222, and/or take any other action associated with imaging device 222. During operation, the user may visualize the video feed from imaging device 222 on an electronic display 198. The real-time signal from imaging device 222 may be processed by control unit 199 and output to the electronic display 198. Electronic display 198 may be one or more electronic displays such as, for example, a monitor, television, tablet, smartphone, portion of control unit 199, virtual reality display, or other display device.

In some examples, an imaging device 222 used at a tip portion of an endoscope, particularly small diameter endoscopes, may be only an image sensor, such as a CCD or CMOS sensor, without any other image processing components and may output an image signal of the raw voltage data acquired by the image sensor. Such a signal may be susceptible to noise while traveling the length of endoscope 101 and noise caused by other environmental considerations. By modulating the raw image signal of the image sensor, the noise may be reduced and the signal may be less susceptible to signal distortion as the signal travels through shaft 108.

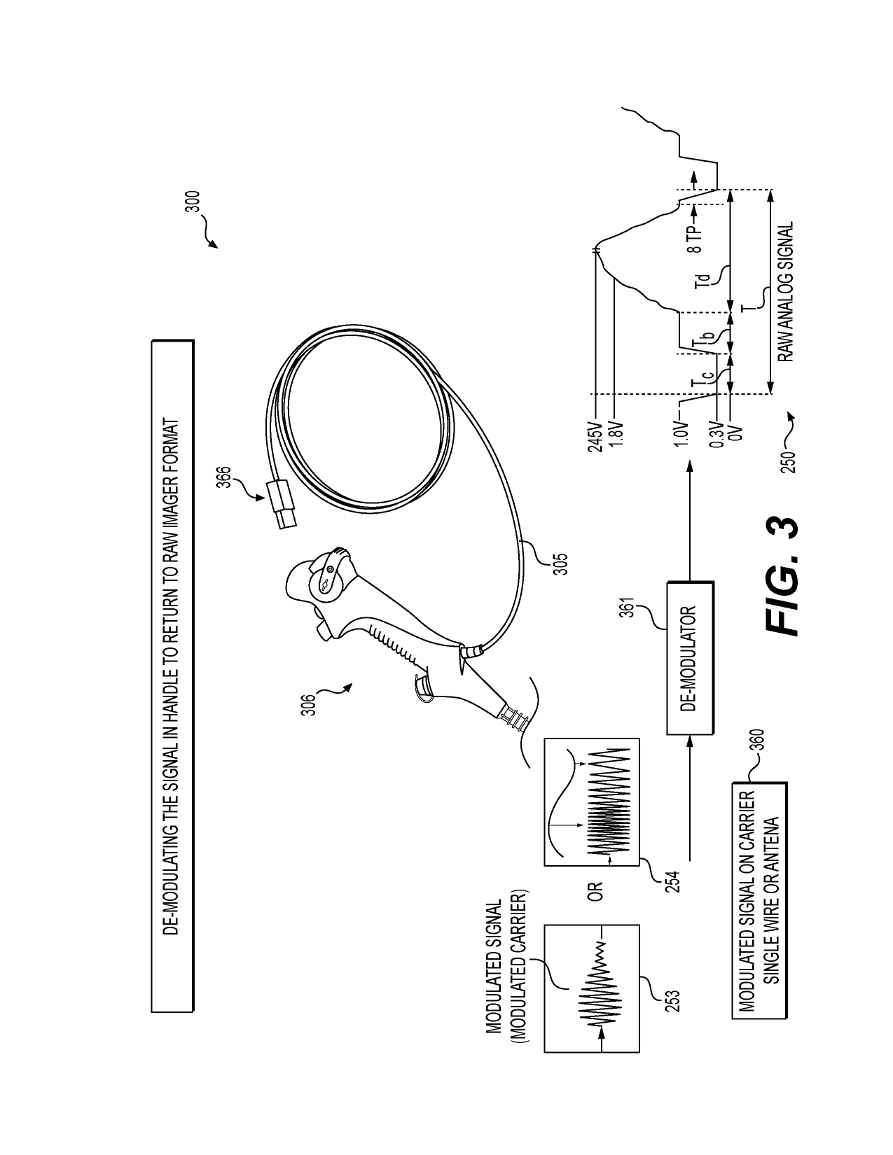

FIG. 2 illustrates a process flow diagram of modulating a raw analog imager signal and sent over a single wire carrier, such as through a single wire extending along the length of a shaft 108, through handle 106, and through umbilicus 105 to control unit 199. As shown in FIG. 2, a raw analog signal 250 may be output from an imaging device 222 at distal portion 119 of endoscope 101. Raw analog signal 250 may be in the form of a voltage signal provided from a CCD image sensor or CMOS sensor, or any other suitable image sensor, and is shown as a graph of voltage output of the sensor over time (T). As an example, shown in the graph of raw analog signal 250 in FIG. 3, values Tc , Tb, Td, and TP are specific to an Omnivision analog sensor. The width of Tc informs the signal processor as to what type of information the following signal is sending. For example, a certain value for Tc could represent that the following signal contains the values for the blue pixels in a row. Tb indicates the black level for that row, Td is the values for the pixels in that row, and TP indicates the end of the row. In some examples, Tc may designate a start pulse or clock period, Tb may designate a “blanking level” period, Td may designate a raw analog period, and TP may be the end period starting with the transition through the blanking level. Other analog sensors may have signals similar to these, may just contain signals like Tc and Td only, or any other combination of these values. Raw analog signal 250 may be output to a modulator 251 within distal portion 119 of shaft 108, to modulate raw analog signal 250.

The process in which one of the characteristic parameters (amplitude, frequency, phase, etc.) of the carrier signal varies linearly with respect to a message signal’s amplitude is called modulation. In the application shown in FIG. 2, the message signal is the raw signal output from imaging device 222, and the carrier signal is the modulated signal 253, 254 outputs from a modulator 251. The carrier technique 252 is applied by modulator 251 to raw analog signal 250 to create a modulated signal 253, 254. It is noted that examples of modulated signals 370,371 are shown as modulated signal waveforms in FIG. 3. There are various forms of modulation, each designed to alter a particular characteristic of the carrier signal wave. The most commonly altered characteristics include amplitude, frequency, phase, pulse sequence, and pulse duration. As shown in FIG. 2, the carrier technique may be amplitude modulation (AM), pulse-amplitude modulation (PAM), pulse-width modulation (PWM), frequency modulation (FM), phase modulation (PM), among any other carrier technique known in the art, such as digital modulation (DM), pulse-coded modulation, frequency-shift keying, or amplitude-shift keying. The modulator 251 within distal tip portion 119 may be any modulator known in the art, such as a sigma-delta modulator, silicon carbide electro-optic modulator, and may be implemented on a circuit board within tip portion 119 of endoscope 101 or any other modulator device known in the art. The type of modulator will depend on the modulation scheme used. For example, a simple FM transmitter would be used for frequency modulated signals. The circuitry is different based on the type of modulation. In some examples, quadrature amplitude modulation (QAM) may be used.

As discussed hereinabove, after raw analog signal 250 is modulated by modulator 251, a modulated signal 253, 254 is output to either a wire extending through shaft 108 and handle 106 to control unit 199, multiple wires extending through endoscope 101, or is output to an antenna or other wireless transmission device to be sent wirelessly to control unit 199, a receiver within handle 106, or any other device for demodulation and processing. In some examples, an antenna or other wireless transmission device may be positioned within tip portion 119, may be positioned within another portion of shaft 108, may be positioned within handle 106, and/or any other portion of medical device system 100. In some examples, one or more wires may couple imaging device 222 and/or modulator 251 to an antenna positioned with shaft 108 or handle 106.

FIG. 3 illustrates a process flow diagram for demodulating a modulated image signal 360, such as a modulated image signal 253, 254 from the process flow diagram shown in FIG. 2. Specifically, FIG. 3 illustrates de-modulating the modulated image signal 253, 254, 360 in handle 306, which in some examples may be handle 106 of endoscope 101, to return the modulated image signal 253, 254 to the raw analog signal 250 of imaging device 222. De-modulator 361 may be positioned within handle 306, 106, and may be any de-modulator known in the art, such as an electronic circuit or a computer program present on one or more processors within handle 106. Any one or more methods for de-modulation may be implemented on the de-modulator 361, such as using a synchronous detector, a frequency modulation demodulator, a phase modulation demodulator, envelope detector, product detector, quadrature detector, Foster-Seeley discriminator, carrier recovery, clock recovery, bit slip, frame synchronization, rake receiver, pulse compression, Received Signal Strength Indication, error detection and correction, any combination of these de-modulation methods and/or other demodulation methods known in the art. After demodulation by de-modulator 361 within handle 306, 106, raw analog signal 250 may be output to control unit 199, for example via wireless transmission, via umbilicus 305, or any other means of transmission. In some examples, de-modulator 361 may be positioned within shaft 108, within umbilicus 105, 305, within a connector 366 of umbilicus 305, or within control unit 199. After de-modulation, raw analog signal 250 may be processed and displayed by control unit 199, for example via outputting a processed image signal to an electronic display 198.

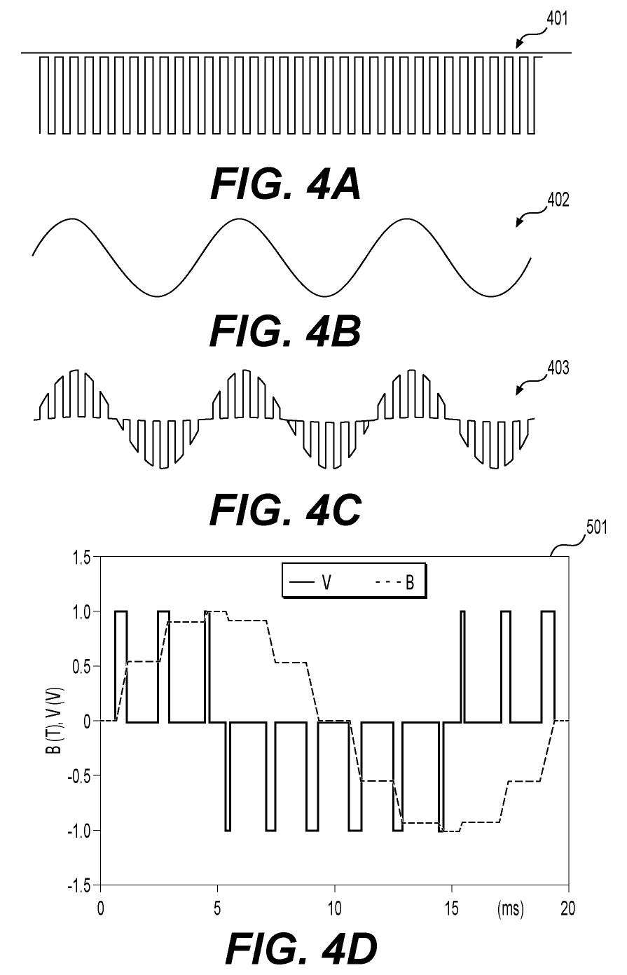

FIGS. 4A-4C illustrate the application of a carrier pulse train 401 (e.g., applied via a modulator 251) to a carrier signal 402, such as raw analog imaging signal 250, to create a pulse amplitude modulated output signal 403. The pulse amplitude modulated output signal 403 may be outputted from modulator 251 to a single wire extending through shaft 108, multiple wires extending through shaft 108, an antenna positioned at tip portion 119, or a wave guide positioned at tip portion 119. In some examples, imaging device 222 (FIG. 2) may output a raw analog signal 250 at a frequency in the 10’s of Megahertz, such as any frequency between 10-99 Megahertz (inclusive), and carrier pulse train 401 may be at a frequency at least 2.5 x higher than the frequency of raw analog signal 250. For example, carrier pulse train 401 may be at a frequency at least between 25-247.5 Megahertz (inclusive); and carrier pulse train 401 may not exceed 5 x higher than the frequency of raw analog signal 250, or may not exceed between 50-495 Megahertz depending on the frequency of raw analog signal 250. The frequency of carrier pulse train 401 may be designed to meet Nyquist’s theorem requirements for sampling while limiting the potential of picking up erroneous noise spikes from the interface connections between imaging device 222 to modulator 251.

FIG. 4D illustrates an example of pulse-width modulation (PWM) including the raw analog signal 250 shown as a solid line (V) and the pulse-width modulated signal shown as a dotted line (B). In pulse-width modulation, the modulated signal may be the integral of the raw analog signal 250. In some examples, pulse-width modulation (PWM) may be used to modified an output load from imaging device 222, which may facilitate the reduction of noise in the output signal as the modulated signal travels through shaft 108.

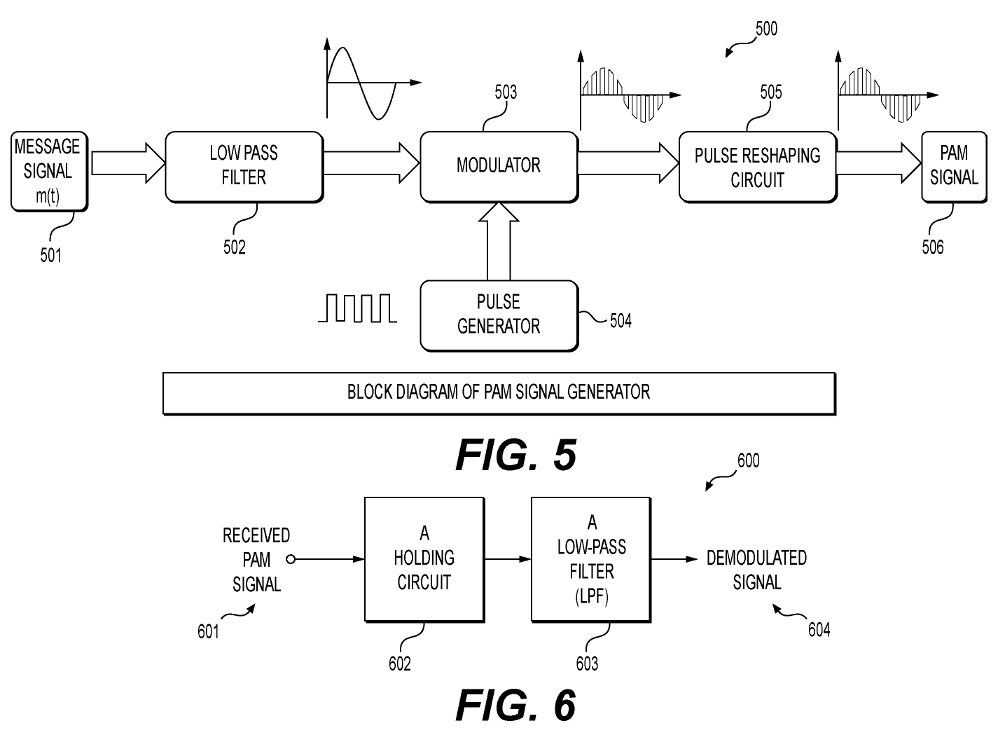

To further reduce noise in a signal from imaging device 222, a low pass filter (LPF) may be incorporated into the signal process flows shown in FIGS. 2 and 3. FIG. 5 illustrates a process flow diagram 500 for imaging device 222 signal modulation incorporating of a low pass filter (LPF) 502. Specifically, the process flow diagram shown in FIG. 5 uses a modulator 503 with a pulse-amplitude modulation technique, with pulse generator 504 used to modulate a message signal 501, for example, a raw data signal from imaging device 222. As shown in FIG. 5, the message signal 501 is first sent to a low pass filter 502 before the signal is sent to modulator 503. Low pass filter 502 may be positioned within tip section 119 of endoscope 101. In other examples, the modulated signal may be sent through shaft 108 and then to a low pass filter positioned in handle 106 or control unit 199. In other examples, the signal process flow may include both low pass filter 502 positioned within tip portion 119 and another low pass filter positioned within handle 106 and/or within control unit 199. Low pass filter 502 may include a signal cutoff just above the highest frequency for imaging device 222 to eliminate any high frequency noise spikes that may be picked up by the system. For example, if the highest output frequency of imaging device 222 is 300 Megahertz, low pass filter 502 may have a cutoff set to 301 Megahertz. In some examples, the system may filter out signals at 88MHz to 108MHz, since that is US FM bands. In some examples, the signal may be sent at a frequency of 915MHz or 868MHz. In some examples, the catheter length (e.g. length of shaft 108) of the device may be a multiple of ¼ wavelength of the carrier frequency, which may facilitate impedance matching.

A pulse reshaping circuit 505 may also be with tip portion 119 and may be used to reshape a modulated signal to a modulated pulse train signal, such as a pulse amplitude modulation (PAM) signal. Referring to FIG. 5, message signal 501 may be output from imaging device 222 to low pass filter 502 (e.g., within tip portion 119), and then the output of low pass filter 502 may be sent to a modulator 503. Pulse generator 504 provides a pulse to modulator 503. A signal from a pulse generator 504 (e.g., at tip portion 119) may be modulated by message signal 501 in modulator 503, and then the output of modulator 503 may be sent to pulse reshaping circuit 505. Pulse reshaping circuit 505 may shape the pulses of the modulated signal to allow a receiver, such as demodulator 361, to easily detect a pulse-amplitude modulated signal 506. Any of the components shown in FIG. 5 may be incorporated in a single circuit board, multiple circuit boards, or any other device known in the art; and may be positioned within tip portion 119 or a portion of shaft 108.

FIG. 6 illustrates a process flow diagram of an exemplary demodulator 600 that may be positioned within handle 106, control unit 199, umbilicus 105, or shaft 106; and may be configured to receive a modulated imaging signal from imaging device 222, for example from the PAM signal generator shown in FIG. 5. The received PAM signal 601, which may be PAM signal 506 sent from pulse reshaping circuit 505, may be received by a holding circuit 602. Holding circuit 602 may include at least one capacitor configured to be charged to the pulse amplitude value of PAM signal 601 and may be configured to hold this amplitude value during the interval between pulses of PAM signal 601. In some examples, holding circuit 602 may be a zero-order holding circuit that considers only the previous sample to decide the value between the two pulses. As shown in FIG. 6, the output of holding circuit 602 may be sent to a low pass filter 603 to smooth a demodulated signal 604. The demodulated signal 604 may then be sent to control unit 199 for processing (e.g., by a processor) and/or display on electronic display 198.

In some examples, the signal from imaging device 222 may be modulated using frequency modulation, such as ultra-broadband frequency modulation. Since the medical device systems described herein, such as medical device system 100, are closed systems with a point to point or direct line of sight link between the transmitter and receiver of imaging signals, a ultra-high broadband frequency modulation carrier technique providing an ultra-wide bandwidth carrier signal may be implemented in the system without concern for adjacent channel interference or broadcast frequency.

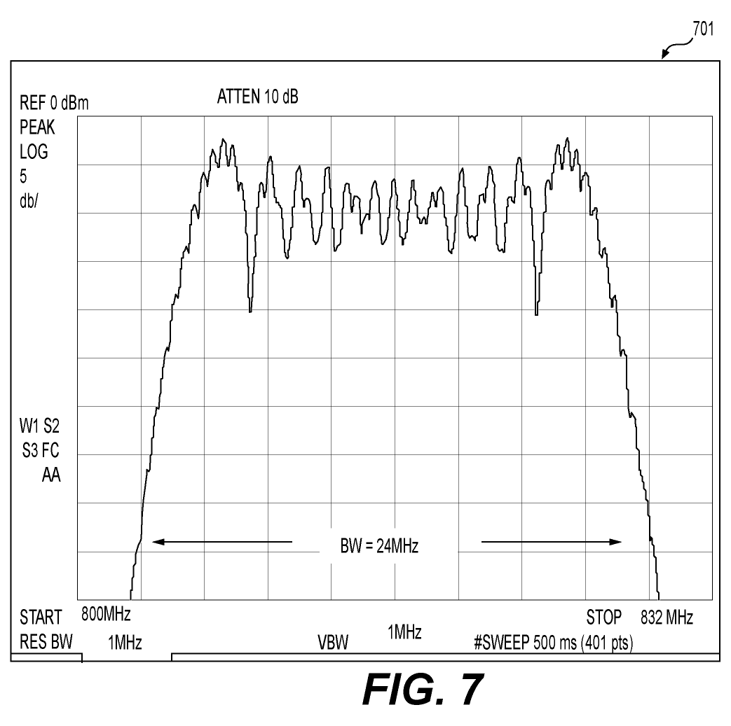

FIG. 7 illustrates a graph 701 of an example of ultra-high broadband frequency modulation spectrum with a high modulation index. As shown in FIG. 7, the bandwidth of the modulated signal may be 24 Megahertz. When implementing frequency modulation in a modulator 251 for an imaging device 222, the carrier technique used by the modulator 251 may implement Carson’s Bandwidth rule for approximate bandwidth requirements for a carrier signal using frequency modulation. In some examples, the imaging device 222 may output an imaging signal with a 4 Megahertz clock system sampled at 8 megahertz, and thus a frequency bandwidth of twice the peak deviation (Δ𝑓 = 4 megahertz) and twice the highest frequency (𝑓𝑚𝑚 = 8 megahertz), the Carson’s Bandwidth rule would need to be a 24 megahertz bandwidth for the ultra-high bandwidth frequency modulated carrier signal. The Carson’s Bandwidth rule may be defined as 𝐶BR = 2(Δ𝑓 + 𝑓𝑚𝑚 ). By utilizing an ultra-high broadband frequency modulation carrier technique, the imaging signal from imaging device 222 may have reduced noise from traveling to handle 106 and/or control unit 199. In addition to reduced noise, the modulation allows for the signal to be transmitted longer distances without signal degradation.

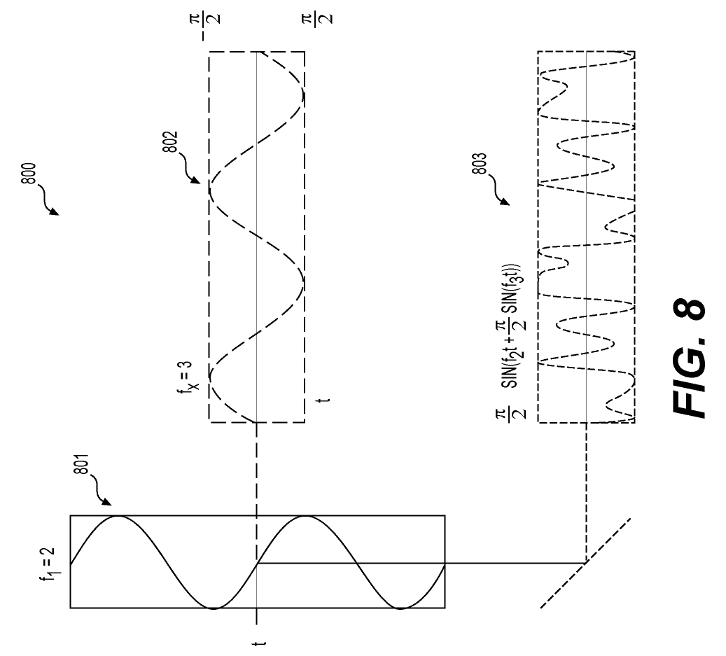

FIG. 8 illustrates an exemplary phase modulation scheme 800 that may be applied using modulator 251 to raw analog signal 250 or any other imaging signal described herein. For phase modulation, the instantaneous amplitude for the analog signal 250 from imaging device 222 modifies the phase of the carrier signal (modulated carrier signal 253) keeping its amplitude and frequency constant. When implementing a phase modulation carrier technique, the phase of modulated carrier signal 253 is modulated to follow the changing signal level (amplitude) of the analog signal 250. The peak amplitude and the frequency of the modulated carrier signal 253 are maintained constant, but as the amplitude of the analog signal 250 changes, the phase of the modulated carrier signal 253 changes correspondingly. As shown in FIG. 8, a modulating waveform 802 is applied to a carrier waveform 801, which may be the raw analog signal 250 from imaging device 222. After modulator 251 applies the modulating waveform 802 to the carrier waveform 801 (e.g., analog signal 250), modulator 251 may output a waveform, for example, a phase modulated waveform 803 of the carrier waveform 801. The phase of the phase modulated waveform 803 may change as the amplitude of the carrier waveform 801 changes. By modulating an analog signal 250 from an imaging device 222, the modulated signal may have reduced noise after transmission compared to transmitting an unmodulated analog signal 250. A carrier signal is represented by c(t) = A_{c}cos(w_{c}t + phi) where w_{c} = 2*pi*frequency, A_{c} is the amplitude, phi is the phase, and t is time. When applying a modulating signal this equation becomes s(t) = A_{c}cos(w_{c}t + kₚm(t)) where m(t) is the message signal (e.g. the analog sensor), and kₚ is the phase sensitivity. If m(t) = Aₘcos(wₘt ) then s(t) = A_{c}cos(w_{c}t)+ Bcos(wₘt)) where B = kₚ*Aₘ = phi. phi is the phase deviation. FIG. 8 is showing an example of the pervious derivation.

In operation of endoscope 101, a user may first actuate imaging button 104 to either initiate a photograph or the start of a video recording using imaging device 222. In some examples, the user may also actuate an illuminator 223 to illuminate the field of view of imaging device 222. The imaging device 222 may then receive photons of light within its field of view at an image sensor of imaging device 222 and may transmit a raw analog signal 250 to a modulator 251 within distal portion 119 of endoscope 101. The modulator 251 may then apply a carrier technique 252 to the raw analog signal 250 to generate a modulated imaging signal 253. As described hereinabove, any carrier technique such as amplitude modulation (AM), pulse-amplitude modulation (PAM), pulse-width modulation (PWM), frequency modulation (FM), phase modulation (PM), or any other carrier technique may be applied to raw analog signal 250. The modulated signal 253, 254 may then be output to one or more wires extending through the length of shaft 108 to handle 106 or may be output to an antenna or other wireless transmission means within tip portion 116, to transmit the modulated signal 253, 254 to handle 106 or control unit 199. Then, the modulated signal 253, 254 may be received by a de-modulator 361 to convert the modulated signal 253 to the analog signal 250. In some examples, the analog signal 250 may then be transmitted from handle 106 to control unit 199 for processing and display by electronic display 198. In other examples, the analog signal 250 may be demodulated and processed at control unit 199 and displayed by electronic display 198.

It will be apparent to those skilled in the art that various modifications and variations may be made in the disclosed systems, devices, and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device system for visualizing internal patient anatomy, comprising:
a shaft including a distal tip portion, the distal tip portion including:
an imaging device;
a signal modulator; and
a pulse generator; and
a control unit operatively coupled to the shaft and including a de-modulator.

2. The medical device system of claim 1, wherein the imaging device is configured to output a first signal to the signal modulator, and wherein the signal modulator is configured to modulate the first signal to output a second signal to the control unit.

3. The medical device system of claim 2, wherein the de-modulator of the control unit is configured to receive the second signal, de-modulate the second signal to output a third signal, and wherein the control unit is configured to output the third signal to an electronic display.

4. The medical device system of claim 3, wherein the second signal is transferred to the de-modulator via a single wire.

5. The medical device system of claim 1, wherein the distal tip portion further comprises a pulse reshaping circuit.

6. The medical device system of claim 2, wherein the control unit further comprises a holding circuit configured to receive the second signal.

7. The medical device system of claim 6, wherein the control unit further comprises a low-pass filter configured to receive the second signal from the holding circuit.

8. The medical device system of claim 3, wherein the second signal is transferred to the de-modulator via an antenna.

9. The medical device system of claim 2, wherein the signal modulator is configured to apply a carrier technique to the first signal to create the second signal, wherein the carrier technique includes at least one of: amplitude modulation (AM), pulse-amplitude modulation (PAM), pulse-width modulation (PWM), frequency modulation (FM), or phase modulation (PM).

10. The medical device system of claim 1, wherein the signal modulator is configured to apply a frequency modulation carrier technique and output a second signal at 24 Megahertz bandwidth.

11. The medical device system of claim 1, wherein the signal modulator is configured to apply a pulse amplitude modulation carrier technique.

12. The medical device system of claim 1, wherein the signal modulator is configured to apply a phase modulation carrier technique.

13. The medical device system of claim 2, wherein the second signal is an integral of the first signal.

14. A medical device system for visualizing internal patient anatomy, comprising:
a shaft including a distal tip portion, the distal tip portion including:
an imaging device;
a signal modulator; and
a pulse generator; and
a handle operatively coupled to the shaft and including a de-modulator.

15. The medical device system of claim 14, wherein the distal tip portion further comprises a low pass filter.

16. The medical device system of claim 14, wherein the distal tip portion comprises a pulse reshaping circuit.

17. A medical device system for visualizing internal patient anatomy, comprising:
a shaft including a distal tip portion, the distal tip portion including:
an imaging device, and
a signal modulator; and
a control unit operatively coupled to the shaft and including a de-modulator;
wherein the imaging device is configured to output a first signal to the signal modulator, wherein the signal modulator is configured to apply a carrier technique to the first signal to create a second signal, wherein the carrier technique includes at least one of: amplitude modulation (AM), pulse-amplitude modulation (PAM), pulse-width modulation (PWM), frequency modulation (FM), or phase modulation (PM).

18. The medical device system of claim 17, wherein the distal tip portion further comprises a pulse generator.

19. The medical device system of claim 18, wherein the distal tip portion further comprises a pulse reshaping circuit.

20. The medical device system of claim 19, wherein the control unit further comprises a holding circuit configured to receive the second signal.
